(19) United States
(12) Patent Application Publication
(10) Pub. No.: US 2023/0392214 A1
(43) Pub. Date: Dec. 7, 2023

(54) Systems and Methods for Assessing Mutational Burden of Neoplasms And Associated Treatments Thereof

(71) Applicant: The Board of Trustees of the Leland Stanford Junior University, Stanford, CA (US)

(72) Inventors: Susanne Tilk, Stanford, CA (US); Dmitri A. Petrov, San Francisco, CA (US); Judith Frydman, Stanford, CA (US); Christina Curtis, Stanford, CA (US)

(73) Assignee: The Board of Trustees of the Leland Stanford Junior University, Stanford, CA (US)

(21) Appl. No.: 18/320,148

(22) Filed: May 18, 2023

Related U.S. Application Data
(60) Provisional application No. 63/364,936, filed on May 18, 2022.

(51) Int. Cl. C12Q 1/6886; A61K 45/06

(52) U.S. Cl. CPC C12Q 1/6886; A61K 45/06; C12Q 2600/154

(57) ABSTRACT

Systems and methods for determining a treatment regimen for a neoplasm or a cancer are provided. A treatment regimen can be based on the mutational burden of the neoplasm or the cancer. When the mutational burden is high, the treatment regimen can include a drug that targets the cellular processes that protect the neoplasm or cancer in response to mutational burden.

### CROSS REFERENCE TO RELATED APPLICATIONS

This application claims priority to U.S. Provisional Application Ser. No. 63/364,936, entitled “Systems and Methods for Assessing Mutational Burden of Neoplasms and Associated Treatments Thereof” filed May 18, 2022, which is incorporated herein by reference in its entirety.

### STATEMENT REGARDING FEDERALLY SPONSORED RESEARCH OR DEVELOPMENT

This invention was made with Government support under contracts CA238296 and GM118165 awarded by the National Institutes of Health. The Government has certain rights in the invention.

### TECHNICAL FIELD

The disclosure is generally directed to systems and methods involving diagnostics and treatments of neoplasms and cancers based upon mutational burden.

### BACKGROUND

Cancer develops from an accumulation of somatic mutations over time. While a small subset of these mutations drives tumor progression, the vast majority of remaining mutations, known as passengers, are believed to not contribute to tumor pathogenicity or progression, despite their abundance and diversity. The number of passengers in a tumor can vary by over four orders of magnitude, even within the same cancer type, from just a few to tens of thousands of point mutations.

### SUMMARY

Various embodiments are directed to diagnostics and treatments of neoplasms and cancers based on mutational load. In several embodiments, genetic material of neoplastic tissue or cancer is assessed for mutational load. In many embodiments, when the genetic material of neoplastic tissue or cancer has a high mutational load, a treatment regimen can be determined. In some embodiments, a treatment is administered based on the mutational load. In some embodiments, a treatment is administered when the mutational load is above a threshold. Treatments include (but are not limited to) transcription inhibitors, cytoskeleton organization inhibitors, protein degradation inhibitors, agonists of apoptosis, chaperone inhibitors, protein inhibitors, DNA replication inhibitors, energy metabolism inhibitors, oxidative stress inhibitors, G-coupled receptor modulators, HSP90 inhibitors, proteasome inhibitors, and ubiquitin-specific proteasome inhibitors.

In some implementations, a method is for determining a treatment regimen for a neoplasm or cancer. The method comprises assessing genetic material of a neoplasm or cancer of an individual to determine a mutational burden. The method comprises based on an amount of mutational burden, determine a treatment regimen.

In some implementations, assessing genetic material comprises quantifying the amount of somatic mutations within the genetic material.

In some implementations, the somatic mutations comprise single nucleotide variations (SNVs), copy number variations (CNVs), insertions, and deletions.

In some implementations, the method further comprises performing a high-throughput sequencing reaction on the genetic material of the neoplasm or the cancer to yield a sequencing result. The method further comprises aligning the sequencing result of the neoplasm or the cancer against a reference genome to identify genetic variations within the genetic material of the neoplasm or the cancer. The genetic variations within the genetic material of the neoplasm or the cancer comprises somatic mutations.

In some implementations, the method further comprises performing a high-throughput sequencing reaction on genetic material of a control sample to yield a sequencing result. The method further comprises aligning the sequencing result of the control sample against a reference genome. The method further comprises aligning the sequencing result of the neoplasm or the cancer with the sequencing result of the control sample to identify somatic variations within the neoplasm or the cancer.

In some implementations, the high-throughput sequencing is whole genome sequencing, whole exome sequencing, or targeted sequencing.

In some implementations, the method further comprises obtaining a biopsy of the individual. The biopsy is a tumor excision, a liquid biopsy, or a biological waste biopsy. The method further comprises extracting the genetic material of the neoplasm or the cancer from the biopsy.

In some implementations, the method further comprises when it is determined that the amount of mutational burden is greater than a threshold, administering to the individual the treatment regimen.

In some implementations, the threshold is a mutational burden in the top 25% of a particular cancer type or is a mutational burden in the top 25% of all cancer types.

In some implementations, the threshold is a mutational burden in the top 10% of a particular cancer type or is a mutational burden in the top 10% of all cancer types.

In some implementations, the threshold is a mutational burden in the top 5% of a particular cancer type or is a mutational burden in the top 5% of all cancer types.

In some implementations, the treatment regimen comprises administration of a HSP90 inhibitor, wherein the HSP90 inhibitor is alvespimycin, BIIB021, CCT018159, ganetespib, gedunin, NVP-AUY922, PU-H71, or VER-49009.

In some implementations, the treatment regimen comprises administration of a proteasome inhibitor, wherein the proteasome inhibitor is bortezomib, carfilzomib, delanzomib, ixazomib, ixazomib-citrate, MG-132, ONX-0914, or oprozomib.

In some implementations, the treatment regimen comprises administration of a ubiquitin-specific proteasome inhibitor, wherein the ubiquitin-specific proteasome inhibitor is NSC-632839, P22077, or P5091.

In some implementations, the neoplasm or the cancer is bile duct cancer, bladder cancer, bone cancer, brain cancer, breast cancer, colon/colorectal cancer, endometrial/uterine cancer, esophageal cancer, gall bladder cancer, gastric cancer, head and neck cancer, kidney cancer, liver cancer, lung cancer, neuroblastoma, ovarian cancer, pancreatic cancer, prostate cancer, rhabdoid tumor, sarcoma, skin cancer, or thyroid cancer.

In some implementations, a method is for assessing cytotoxicity of a compound on a neoplastic cell, a cancer cell, or a tumor cell with high mutation burden. The method comprises performing a high-throughput sequencing reaction on genetic material of a specimen to yield a sequencing result. The specimen is a growth of neoplastic cells, a growth of cancer cells, or a tumor. The method comprises quantifying the amount of somatic mutations within the genetic material. The method comprises determining that the specimen has a mutational burden that is greater than a threshold. The method comprises contacting a neoplastic cell of the growth of neoplastic cells, a cancer cell of the growth of neoplastic cells, or a tumor cell of the tumor with a compound to assess the cytotoxicity of the compound on the neoplastic cell, the cancer cell, or the tumor cell.

In some implementations, the neoplastic cell, the cancer cell, or the tumor cell is in vitro.

In some implementations, the neoplastic cell, the cancer cell, or the tumor cell is in vivo.

In some implementations, the compound is classified as: a transcription inhibitor, a cytoskeleton organization inhibitor, a protein degradation inhibitor, an agonist of apoptosis, a chaperone inhibitor, a protein inhibitor, a DNA replication inhibitor, an energy metabolism inhibitor, an oxidative stress inhibitor, a G-coupled receptor modulator, a HSP90 inhibitor, a proteasome inhibitor, or a ubiquitin-specific proteasome inhibitor.

In some implementations, the somatic mutations comprise at least one of: single nucleotide variations (SNVs), copy number variations (CNVs), insertions, or deletions.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides circular bar plots depicting protein complexes from the CORUM database (left) and pathways from the KEGG database (right) that are significantly enriched (p<0.05) in response to mutational load. Length of bars denote negative log 10 of adjusted p-value and colors denote broad functional groups enriched in both databases.

FIG. 2 provides a flow chart of an example of a method for determining a treatment regimen based on mutational burden.

FIG. 3 provides heat maps showing no collinearity of point mutations and copy number alterations in human tumors (TCGA) and cancer cell lines (CCLE). Heatmap of Pearson's correlation coefficients between different classes of mutations in CCLE (cancer cell lines) and TCGA (human tumors). Shades denote magnitude of correlation coefficients and whether the relationship is positive, negative, or negligible. CNAs are defined as the combined number of amplifications and deletions, while SNVs are the combined number of all point mutations.

FIG. 4 provides a schematic depicting an overview of the GLMM used to measure the association of mutation load with gene expression while controlling for potential co-variates (purity and cancer type). Genes with a significant, positive β₁ regression coefficient and false discovery rate (FDR)<0.05 are used for gene set enrichment analysis.

FIG. 5 provides bar plots of protein complexes from the CORUM database (left) and pathways from the KEGG database (right) that are significantly enriched (p<0.05) in response to mutational load. Length of bars denote negative login of adjusted p-value and colors denote broad functional groups enriched in both databases.

FIGS. 6A and 6B provide data showing that genes significantly expressed from the transcriptional screen mostly fall into the upper quartile of effect sizes, which are enriched for proteostasis complexes. FIG. 6A: Volcano plot of positive β₁ regression coefficients and negative log₁₀ adjusted p-values measuring the association of mutation load and the expression of individual genes from the transcriptional screen in FIG. 4. Genes that are significantly expressed from the transcriptional screen mostly fall into the upper quartile. FIG. 6B: Barplot of significant protein complexes in the CORUM database identified using gene set enrichment analysis only on genes that fall into the upper quartile of effect sizes. Genes in the upper quartile of effect sizes contain half of the genes that were identified as significant previously (n=2,152 vs n=5,330), yet still identify protein degradation, translation and chaperones as the top significant protein complexes.

FIG. 7A provides counts of the number of under-expressed transcripts with intron retention events, relative to counts of all intron retention events in tumors binned by the total number of protein-coding mutations. Intron retention events with PSI>80% are counted. Error bars are 95% confidence intervals determined by bootstrap sampling.

FIGS. 7B and 7C provide data showing intron retention events that overlap with mutations do not account for the association of gene silencing in high mutational load tumors. FIG. 7B: Counts of the number of intron retention events filtered due to overlap with a mutation present in the same gene (and thus corresponding to potential eQTLs) compared the number of remaining alternative splicing events with no overlap with a mutation. Alternative splicing events filtered represent ˜1% of all alternative splicing events across all tumors. FIG. 7C: Counts of the number of under-expressed transcripts with intron retention events, relative to counts of all intron retention events in tumors binned by the total number of protein-coding mutations. Shown are when trends when (left panel) not filtering alternative splicing events due to overlap with mutations and (right panel) when events are filtered (same as FIG. 7A). Intron retention events with PSI>80% are counted. Error bars are 95% confidence intervals determined by bootstrap sampling. These results further support the prediction that gene silencing is elevated in high mutational load tumors and likely mediated by the coupling of intron retention with mRNA decay.

FIG. 7D provides data showing the number of under-expressed transcripts increases with the mutational load of tumors for different PSI value thresholds and alternative splicing events. Left panel: Counts of the number of under-expressed transcripts with intron retention events, relative to counts of all intron retention events in tumors binned by the total number of protein-coding mutations. Intron retention events with different PSI thresholds are shown. Right panel: Counts of the number of under-expressed transcripts that contain different classes alternative splicing events, relative to counts of all alternative splicing events of the same class in tumors binned by the total number of protein-coding mutations. Alternative splicing events of different classes are shown colored (AA=Alternate Acceptor Sites, AD=Alternate Donor Sites, AP=Alternate Promoter, AT=Alternate Terminator, ES=Exon Skip, ME=Mutually Exclusive Exons, RI=Retained Intron). Error bars are 95% confidence intervals determined by bootstrap sampling.

FIG. 7E provides a barplot of significant protein complexes in the CORUM database and Reactome pathway database with more (bottom) and less (top) intron retention events in high mutational load tumors compared to low mutational load tumors.

FIGS. 8A and 8B provide data showing protein folding, degradation, and synthesis are regulated in both high mutational load tumors (TCGA) and cell lines (CCLE). Box plots of β₁ regression coefficients (FIG. 8A) and negative log₁₀ adjusted p-values (FIG. 8B) measuring the association of mutation load and the expression of individual genes in chaperone, proteasome, and ribosome complexes. Shown are regression coefficients from human tumors (TCGA) on the left and cell lines (CCLE) on the right. Percentages and grey lines on top panels show the quantile distribution of regression coefficients.

FIG. 9A provides data showing the association between expression in proteostasis complexes and mutational load is not driven by a single cancer type in TCGA. Box plots of regression coefficients from the GLMM measuring the association of the expression of each individual gene with the mutational load of tumors in TCGA colored by different proteostasis complexes. Shown are regression estimates after removing each individual cancer type (x-axis) and re-running the GLMM. Cancers types: ACC, BLCA, BRCA, CESC, COA, DLBC, GBM, KICH, KIRC, KIRP, LAMI, LGG, LIHC, LUSC, MESO, OV, PCPG, PRAD, READ, SARC, STAD, THCA, THYM, UCEC, UCS, and UVM.

FIG. 9B provides data showing linear regression analysis within cancer types in TCGA captures similar expression responses to mutational load across proteostasis complexes. Heatmap of β₁ regression coefficients measuring the effect of mutational load on gene expression in proteostasis complexes while controlling for tumor purity within cancer types which have enough samples to accurately measure effect sizes (N>150) and contain a sufficiently large enough mutational load to potentially generate a proteostasis response (median protein coding mutations >25). ‘MutLoad’ shows log 10 of the median number of protein coding mutations for each cancer type.

FIG. 9C provides data showing association between the expression in proteostasis complexes and mutational load is not driven by patient age. Boxplots of regression coefficients from the GLMM measuring the association of the expression of each individual gene with the mutational load of tumors from TCGA colored by different proteostasis complexes. Shown are regression coefficients when running the GLMM on tumors stratified by different age groups (x-axis).

FIG. 10A provides data showing association between the expression in proteostasis complexes and mutational load is not driven by a single cancer type in CCLE. Box plots of regression coefficients from the GLM measuring the association of the expression of each individual gene with the mutational load of tumors colored by different proteostasis complexes. Shown are regression estimates after removing each cancer type in CCLE (x-axis) and re-running the GLM. Cancer types: bile duct cancer, bladder cancer, bone cancer, brain cancer, colon/colorectal cancer, endometrial/uterine cancer, esophageal cancer, fibroblast, gastric cancer, head and neck cancer, kidney cancer, leukemia, liposarcoma, liver cancer, lung cancer, lymphoma, neuroblastoma, ovarian cancer, pancreatic cancer, rhabdoid, and skin cancer.

FIG. 10B provides data showing similar patterns of expression and protein abundances in response to mutational load in CCLE within genes that regulate protein folding, degradation, and synthesis. Box plots of β₁ regression coefficients measuring the association of mutation load and protein abundance (right) or gene expression (left) of individual genes in chaperone, proteasome, and ribosome complexes. Shown are regression coefficients from cancer cell lines (CCLE), which contains the largest dataset available of RNA (n=1377) and protein (n=373) abundances which are harmonized across samples. Percentages and grey lines on top panels show the quantile distribution of regression coefficients measuring the association of mutational load and expression for all genes in the genome within each dataset.

FIGS. 11A and 11B provide heat maps showing viability in high mutational load cell lines decreases when proteostasis machinery is disrupted. FIG. 11A: Heatmap of β₁ regression coefficients jointly measuring the association of mutational load and cell viability after expression knockdown of individual genes in proteostasis complexes. FIG. 11B: Heatmap of β₁ regression coefficients measuring the association mutational load and cell viability after inhibition of proteostasis machinery via drugs. Both panels show how stable regression estimates are when including all cancer types (‘All Cancers’) shown in black boxes and when removing each individual cancer type on the y-axis. Stars denote whether the relationship is significant (*=p<0.05; **=p<0.005; ***=p<0.0005).

FIGS. 12A and 12B provide data showing targeting proteostasis machinery is a key vulnerability in high mutational load cell lines. FIG. 12A: Bar plot of the number of drugs in the PRISM database significantly (black) and not significantly (grey) associated with mutational load and cell viability using a simple generalized linear model (GLM). FIG. 12B: Fraction of drugs in broad functional categories significantly negatively associated with mutational load and cell viability from the GLM. Confidence intervals were determined by randomly sampling 50 drugs in each functional category 100 times. Dashed line is the median of randomly sampled drugs across all categories.

### DETAILED DESCRIPTION

Turning now to the drawings and data, systems and methods for assessing neoplasms and cancers for determining a treatment regimen are provided. It is now understood that high mutational burden within neoplastic tissue or cancer is a vulnerability that can be exploited. Mutational burden is an amount of somatic mutations within the genetic material of a neoplasm or cancer. In several embodiments, the genetic material of a neoplasm or cancer is assessed for mutational burden. In many embodiments, a treatment regimen is determined based on the mutational burden. For instance, a neoplasm or cancer with high mutational burden can be treated with particular drugs that target the cellular processes that protect the neoplasm or cancer in response to mutational burden. In some embodiments, a neoplasm or a cancer is treated based on the determined treatment regimen

Whether passenger mutations are damaging to tumors has long been a matter of debate. Generally, many in the art suggest that passengers are functionally unimportant to tumors given that most non-synonymous mutations are not removed by negative selection in somatic tissues. This is in direct contrast to the human germ-line, where non-synonymous mutations do appear to be functionally damaging in most genes and the signals of negative selection are pervasive. The common explanation for why the protein-coding mutations are removed in the human-germline but maintained in somatic tissues is that most genes are only important for multi-cellular function at the organismal level (e.g. during development), but not during somatic growth.

Here, an alternative explanation is provided that suggests non-synonymous mutations are indeed damaging in somatic evolution, but negative selection is too inefficient at removing them due to the linkage effects driven by the lack of recombination in somatic cells. Without recombination to break apart combinations of mutations, the beneficial drivers and deleterious passengers that happen to be present in the same genome are acted upon by selection together. This makes it less efficient for selection to favor the beneficial drivers and to remove the deleterious passengers. As a result, a substantial number of weakly damaging passengers can accrue in neoplasms and cancer due to inefficient negative selection over time.

If individual passengers are in fact substantially damaging in cancer, successful tumors with thousands of linked mutations must find ways to maintain their viability by mitigating the deleterious effects resulting from mutational burden. While paths to mitigation are difficult to predict for non-coding mutations, tumors with mutations in protein-coding genes are expected to minimize the damaging phenotypic effects of protein mis-folding stress. To investigate this hypothesis, gene expression was used to assess how the physiological state of cancer cells change as they accumulate protein coding mutations. Using a general linear mixed effects regression model (GLMM) and leveraging variation across 10,295 tumors from 33 cancer types, it was found that complexes that re-fold proteins (chaperones), degrade proteins (proteasome) and splice mRNA (spliceosome) are more up-regulated in neoplasms and cancers with higher mutation burdens. The results were validated by showing that similar physiological responses occur in high mutational burden cancer cell lines as well. These results establish a new diagnostic and treatment regimen for neoplasms and cancers with higher mutation burdens.

### Assessment of Neoplastic Growth and Cancers Via Mutational Burden

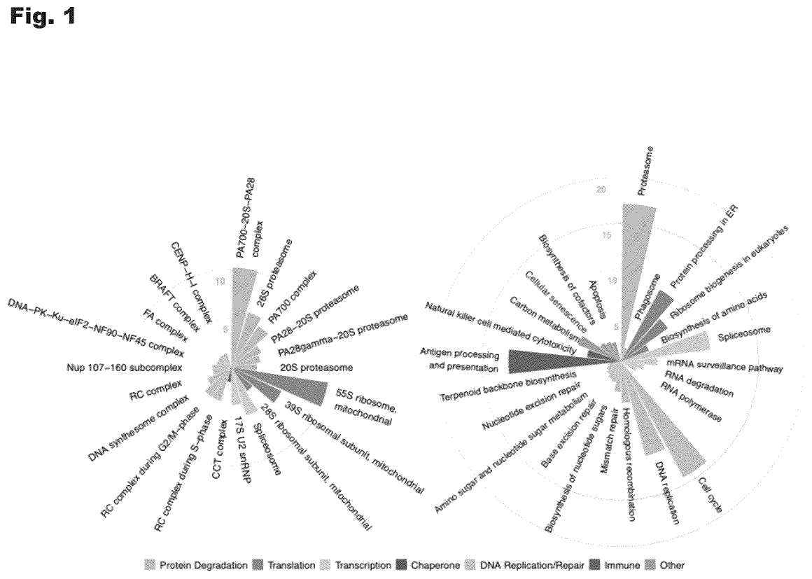

A number of embodiments are directed to assessment of neoplasms and cancers based on their mutational burden and determining an appropriate treatment regimen. In several embodiments, genetic material of a neoplasm or cancer is assessed for mutational burden. Neoplasms and cancers with higher mutational burden are suspect to treatments that target the biological processes that allow for the high mutational burden. FIG. 1 provides a schematic of protein complexes (FIG. 1 left) and pathways (FIG. 1 right) that are more upregulated in response to greater mutational burden. Accordingly, a treatment regimen that targets these protein complexes and/or pathways can be utilized for neoplasms and cancers with high burden.

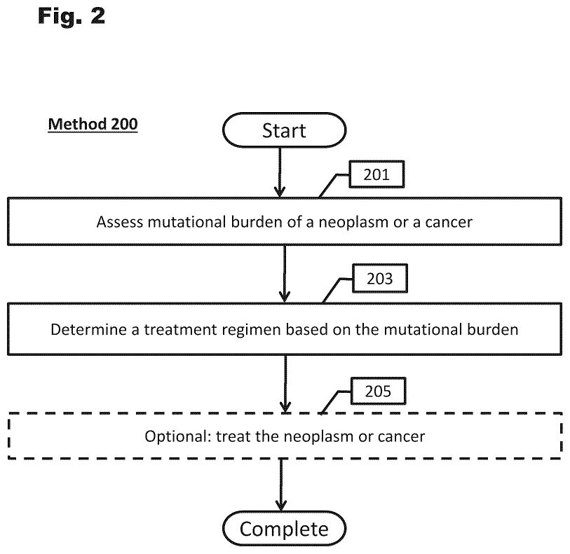

Several embodiments are directed to methods for assessing a neoplasm or cancer. Generally, genetic material of the neoplasm or cancer is examined for mutational burden. A neoplasm or cancer with high mutational burden can be treated with drugs that target the protein complexes and/or pathways that help mitigate the negative effects of mutational burden. Provided in FIG. 2 is an exemplary method to assess a neoplasm or cancer to determine a treatment regimen, which can be used to treat the neoplasm or cancer.

Method 200 begins with assessing 201 mutational burden of a neoplastic cell, a cancer cell, a tumor, a neoplasm, or a cancer. In several embodiments, genetic material of the neoplastic cell, the cancer cell, the tumor, neoplasm, or the cancer is examined for mutational burden. In some embodiments, a biopsy of an individual is utilized to obtain genetic material for examination. Various biopsies can be utilized to extract genetic material, including (but not limited to) tumor excision, liquid biopsy (e.g., blood draw), or biological waste biopsy. In some embodiments, the genetic material is obtained from a neoplastic cell, a cancer cell, or a tumor grown in vitro or in vivo. The genetic material is any nucleic acid that would be able to provide analysis of mutational burden, including (but not limited to) DNA, RNA, cell-free nucleic acids, or any other nucleic acids that would have derived from the neoplasm or cancer.

Mutational burden is assessed by quantifying somatic mutations from the genetic material. Mutations that can be assessed include (but are not limited to) single nucleotide variations (SNVs), copy number variations (CNVs), insertions, and deletions. In some embodiments, a total number of mutations are quantified. In some embodiments, a relative number of mutations are quantified, such as (for example) the number of mutations per kilobase of genetic material.

Various methodologies can be utilized to quantify mutations. Generally, the genetic material is sequenced (e.g., high-throughput sequencing) and the sequencing result is assessed against a control sequence to identify somatic mutations across a genome, an exome, or a targeted set of sequence fragments. In some embodiments, whole genome sequencing is performed. In some embodiments, whole exome sequencing is performed. In some embodiments, targeted sequencing of a high number of sequence fragments is performed (e.g., over 1000 sequence fragments, over 10,000 sequence fragments, over 100,000 sequence fragments, or over 1,000,000 sequence fragments). In some embodiments, the control sequence is genetic material of healthy tissue (i.e., non-neoplasm or non-cancer) of the individual. In some embodiments, the control sequence is an established reference sequence. Reference sequences include (but are not limited to) hg19, hg38, and population genomes or superpopulation genomes such as those from the 1000 Genomes project (Clarke, et al. Nat Methods 9, 459-462 (2012), the disclosure of which is incorporated herein by reference).

Various methodologies can be utilized to identify mutations. Generally, the sequencing result of genetic material is aligned to yield a genome, an exome, or a targeted portion of the genome. Alignment can be done using an established reference sequence, such as (for example) hg19, hg38, and population genomes or superpopulation genomes such as those from the 1000 Genomes project. Mutations can be called based on variation from the control sequence. Protein coding mutations can further be assessed based on the effect of the variation on the protein sequence.

Method 200 further determines (203) a treatment regimen based on the mutational burden. It has been determined that neoplasms and cancers with higher mutational burden are susceptible to drugs that counteract the cellular processes that help mitigate the effects of somatic mutation.

In several embodiments, cancers and neoplasms with high mutational burden are determined to benefit from drugs that modulate the protein structures cellular processes that are more upregulated with greater mutational burden. Accordingly, in various embodiments, drug treatments include (but are not limited to) transcription inhibitors, cytoskeleton organization inhibitors, protein degradation inhibitors, agonists of apoptosis, chaperone inhibitors, protein inhibitors, DNA replication inhibitors, energy metabolism inhibitors, oxidative stress inhibitors, G-coupled receptor modulators, HSP90 inhibitors, proteasome inhibitors, and ubiquitin-specific proteasome inhibitors.

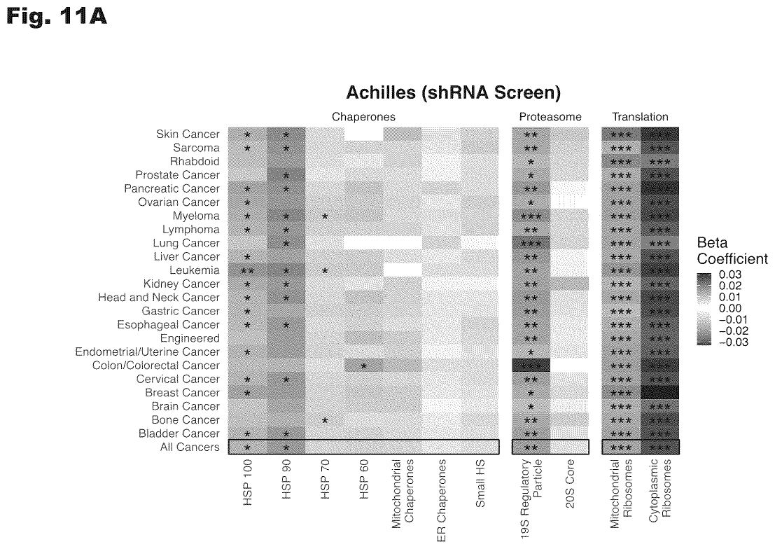

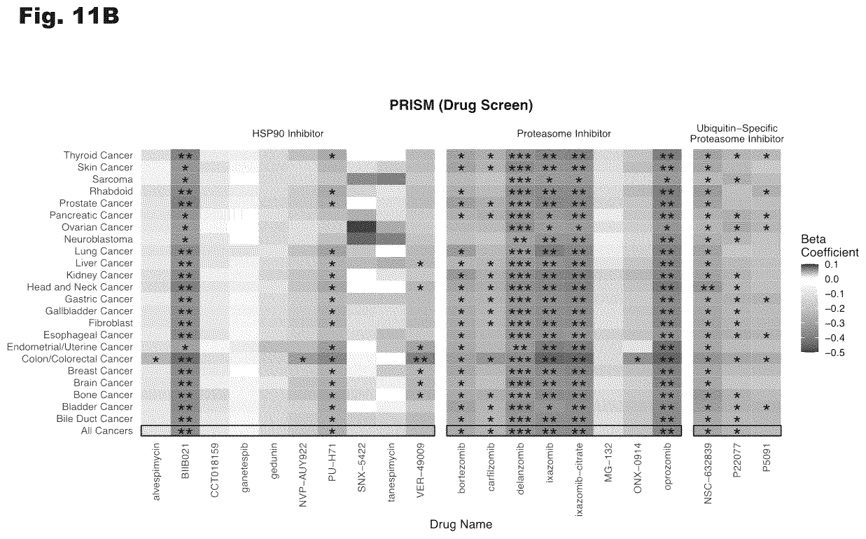

In some embodiments, the drug for treatment is a HSP90 inhibitor. HSP90 inhibitors include (but are not limited to) alvespimycin, BIIB021, CCT018159, ganetespib, gedunin, NVP-AUY922, PU-H71, and VER-49009. For more on each drug's effectiveness on cancer type, see FIG. 11B.

In some embodiments, the drug for treatment is a proteasome inhibitor. Proteasome inhibitors include (but are not limited to) bortezomib, carfilzomib, delanzomib, ixazomib, ixazomib-citrate, MG-132, ONX-0914, and oprozomib. For more on each drug's effectiveness on cancer type, see FIG. 11B.

In some embodiments, the drug for treatment is a ubiquitin-specific proteasome inhibitor. Ubiquitin-specific proteasome inhibitors include (but are not limited to) NSC-632839, P22077, and P5091. For more on each drug's effectiveness on cancer type, see FIG. 11B.

In some embodiments, the drug for treatment is a growth factor inhibitor, an apoptosis agonist, an energy metabolism inhibitor, an inflammatory/immune modulator, a protein synthesis inhibitor, a DNA replications inhibitor, a cytoskeleton inhibitor, a transcription inhibitor, an ion channel regulator, a protein degradation inhibitor, a chaperone inhibitor, an oxidative stress activator, a lipid metabolism inhibitor, a growth hormone inhibitor, an angiogenesis inhibitor, a neurotransmitter inhibitor, a neurotransmitter enhancer, an oxidative stress inhibitor, a mucolytic agent, a melanin inhibitor, a histone/methylation inhibitor, a sugar metabolism inhibitor, a G-coupled protein receptor regulator, a protein metabolism inhibitor, a nitrogen metabolism inhibitor, or a viral replication inhibitor. Non-limiting examples of drugs for treatment is provided in Table 1.

It is to be understood that drug combinations can also be provided. Accordingly, two or more drugs are provided for treatment. Any two drugs described herein can be combined together.

In several embodiments, the treatment regimen is based on the amount of mutational burden. In some embodiments, the drugs described herein for treatment are to be utilized within a regimen when the mutational burden is above a threshold. Any appropriate threshold can be utilized. In some embodiments, neoplasms or cancers having mutational burden in the top 5% of the particular cancer or in the top 5% of all cancers are provided a treatment regimen described herein. In some embodiments, neoplasms or cancers having mutational burden in the top 10% of the particular cancer or in the top 10% of all cancers are provided a treatment regimen described herein. In some embodiments, neoplasms or cancers having mutational burden in the top 25% of the particular cancer or in the top 25% of all cancers are provided a treatment regimen described herein. Table 2 provides mutational burden counts for the top 5%, top 10%, and top 25% in various particular cancers or of all cancer types.

Based upon the mutational burden and determined treatment regimen, a neoplastic cell, a cancer cell, a tumor, a neoplasm, or a cancer is optionally treated (205). Accordingly, the determined treatment regimen can be administered, such as the treatment regimens described herein.

In some embodiments, when a neoplastic cell, a cancer cell, a tumor, a neoplasm, or a cancer has a mutational burden over a threshold, the neoplastic cell, a cancer cell, a tumor, a neoplasm, or a cancer is contacted with a transcription inhibitor, a cytoskeleton organization inhibitor, a protein degradation inhibitor, an agonist of apoptosis, a chaperone inhibitor, a protein inhibitor, a DNA replication inhibitor, an energy metabolism inhibitor, an oxidative stress inhibitor, a G-coupled receptor modulator, a HSP90 inhibitor, a proteasome inhibitor, or a ubiquitin-specific proteasome inhibitor. In some embodiments, when a neoplastic cell, a cancer cell, a tumor, a neoplasm, or a cancer has a mutational burden is not over a threshold, the neoplastic cell, a cancer cell, a tumor, a neoplasm, or a cancer is not contacted with a transcription inhibitor, a cytoskeleton organization inhibitor, a protein degradation inhibitor, an agonist of apoptosis, a chaperone inhibitor, a protein inhibitor, a DNA replication inhibitor, an energy metabolism inhibitor, an oxidative stress inhibitor, a G-coupled receptor modulator, a HSP90 inhibitor, a proteasome inhibitor, or a ubiquitin-specific proteasome inhibitor, but is instead contacted with a medicament of a standardized treatment protocol for the cancer type.

In some embodiments, when a neoplastic cell, a cancer cell, a tumor, a neoplasm, or a cancer has a mutational burden over a threshold, an individual in which the neoplastic cell, a cancer cell, a tumor, a neoplasm, or a cancer was derived from is administered a treatment comprising a transcription inhibitor, a cytoskeleton organization inhibitor, a protein degradation inhibitor, an agonist of apoptosis, a chaperone inhibitor, a protein inhibitor, a DNA replication inhibitor, an energy metabolism inhibitor, an oxidative stress inhibitor, a G-coupled receptor modulator, a HSP90 inhibitor, a proteasome inhibitor, or a ubiquitin-specific proteasome inhibitor. In some embodiments, when a neoplastic cell, a cancer cell, a tumor, a neoplasm, or a cancer has a mutational burden is not over a threshold, an individual in which the neoplastic cell, a cancer cell, a tumor, a neoplasm, or a cancer was derived from is not administered a treatment comprising a transcription inhibitor, a cytoskeleton organization inhibitor, a protein degradation inhibitor, an agonist of apoptosis, a chaperone inhibitor, a protein inhibitor, a DNA replication inhibitor, an energy metabolism inhibitor, an oxidative stress inhibitor, a G-coupled receptor modulator, a HSP90 inhibitor, a proteasome inhibitor, or a ubiquitin-specific proteasome inhibitor, but is instead administered a medicament of a standardized treatment protocol for the cancer type.

Neoplasm and cancer types that can be treated include (but are not limited to) all cancers generally, bile duct cancer, bladder cancer, bone cancer, brain cancer, breast cancer, colon/colorectal cancer, endometrial/uterine cancer, esophageal cancer, gall bladder cancer, gastric cancer, head and neck cancer, kidney cancer, liver cancer, lung cancer, neuroblastoma, ovarian cancer, pancreatic cancer, prostate cancer, rhabdoid tumor, sarcoma, skin cancer, and thyroid cancer.

Dosing and therapeutic regimens can be administered appropriate to the cancer to be treated, and can be determined by preclinical and clinical studies.

In some embodiments, drugs are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size.

A therapeutically effective amount can be an amount sufficient to prevent reduce, ameliorate or eliminate the symptoms of breast cancer. In some embodiments, a therapeutically effective amount is an amount sufficient to reduce the growth and/or metastasis of a cancer.

While specific examples of methods for assessing mutational burden and determining a treatment regimen are described above, one of ordinary skill in the art can appreciate that various steps of the method can be performed in different orders and that certain steps may be optional according to some embodiments of the disclosure. As such, it should be clear that the various steps of the process could be used as appropriate to the requirements of specific applications.

### Exemplary Embodiments

The embodiments of the disclosure will be better understood with the several examples provided. Validation results are also provided.

Cancers Adapt to their Mutational Load by Buffering Protein Misfolding Stress

In this example, the ability of tumors to maintain their viability by mitigating the detrimental effects of mutational load was examined by analyzing tumor tissues with paired mutational and gene expression profiles to assess how the physiological state of cancer cells change as they accumulate protein coding mutations. Using a general linear mixed effects regression model (GLMM), variation across 10,295 tumors from 33 cancer types was leverage and found that complexes that re-fold proteins (chaperones), degrade proteins (proteasome) and splice mRNA (spliceosome) are up-regulated in high mutation load tumors. These results were validated by showing that similar physiological responses occur in high mutational load cancer cell lines as well. Finally, a causal connection was established by showing that high mutational load cell lines are particularly sensitive when proteasome and chaperone function is disrupted through downregulation of expression via short-hairpin RNA (shRNA) knock-down or targeted therapies. Collectively, these data indicate that the viability of high mutational load tumors is strongly dependent on the up-regulation of complexes that degrade and refold proteins, revealing a generic vulnerability of cancer that can be therapeutically exploited.

### Quantifying Transcriptional Response to Mutational Load in Human Tumors

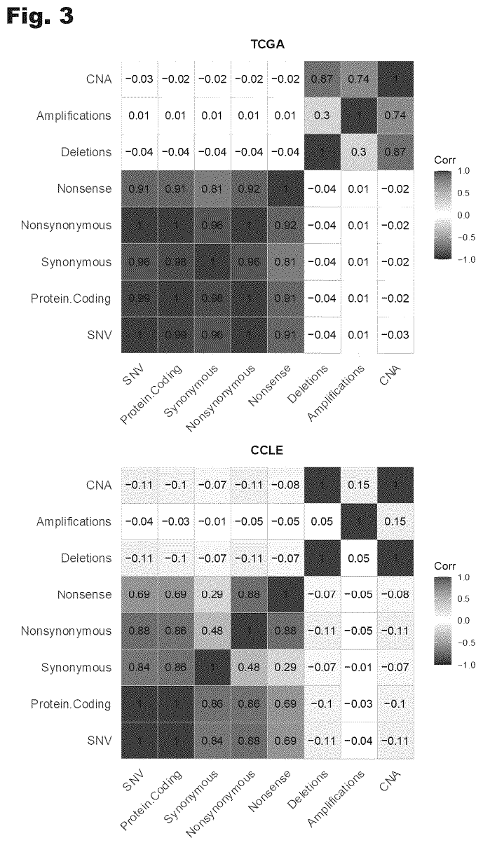

A genome-wide screen was performed to systematically identify which genes are transcriptionally upregulated in response to mutational load in human tumors. To do so, publicly available whole-exome and gene expression data from 10,295 human tumors across 33 cancer types from The Cancer Genome Atlas (TCGA) were assessed. Multiple classes of mutations were considered to define mutational load and investigated their degree of collinearity, focusing on protein-coding regions since the use of whole-exome data limits the ability to accurately assess mutations in non-coding regions. It was found that there is a high degree of collinearity among synonymous, non-synonymous and nonsense point mutations in protein coding genes (R>0.9) but weak collinearity between point mutations and copy number alterations (R<0.05) (FIG. 3). Thus, it was decided to focus on the aggregate effects of protein-coding mutations and for all analyses defined mutational load as login of the total number of point mutations in protein-coding genes. For simplicity, all mutations were used rather than focusing only on passenger mutations since identifying genuine drivers against a background of linked passenger events can be difficult, especially for tumors with many mutations.

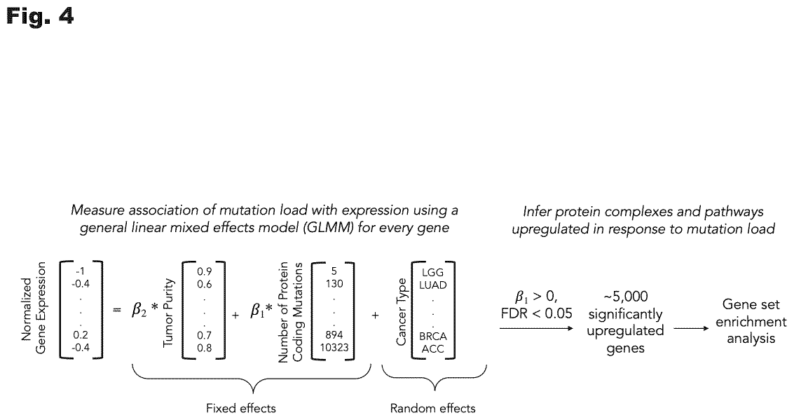

Since gene expression can vary across tumors due to many factors, such as cancer type, tumor purity and other unknown factors, a generalized linear mixed model (GLMM) was utilized to measure the association of mutational load and gene expression while accounting for these potential confounders (FIG. 4). Within the GLMM, tumor purity and mutational load were modeled as fixed effects whereas cancer type was modeled as a random effect since it varies across groups of patients and can be interpreted as repeated measurements across groups. The following GLMM was applied separately to each gene,

Y˜β₀+β₁X₁+β₂X₂+v+e

where Y is a vector of normalized expression values across all tumors, β₀ is the fixed intercept, β₁ is the fixed slope for the predictor variable X₁ which is a vector of mutational load values for each tumor, β₂ is the fixed slope for the predictor variable X₂ which is a vector of the purity of each tumor, v is the random intercept for each cancer type, and e is a Gaussian error term (for more details, see Methods section below).

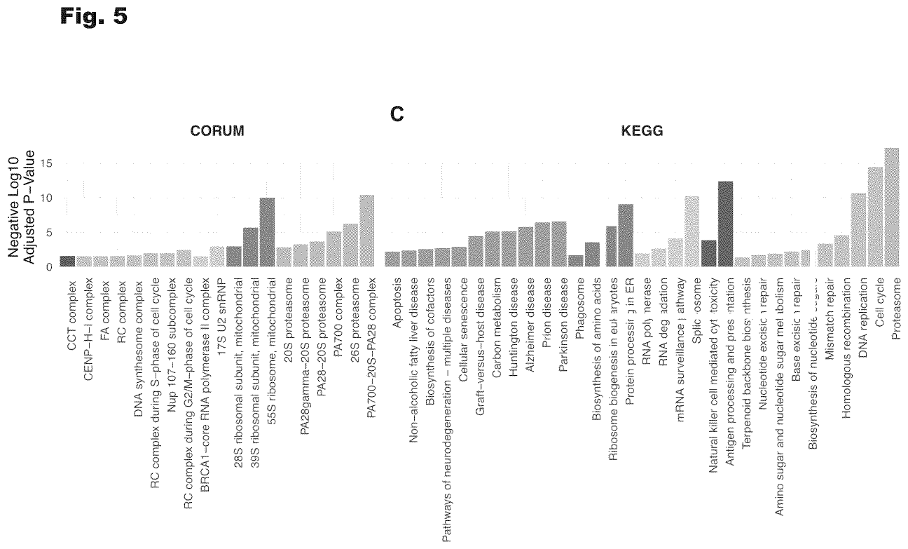

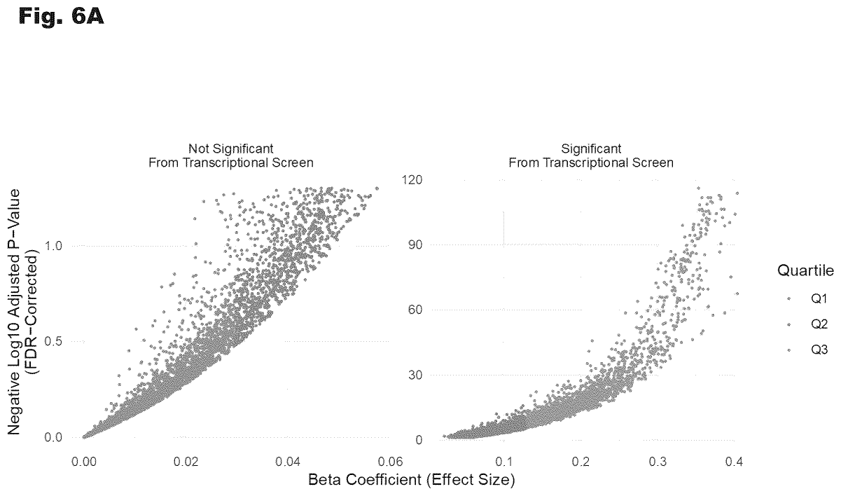

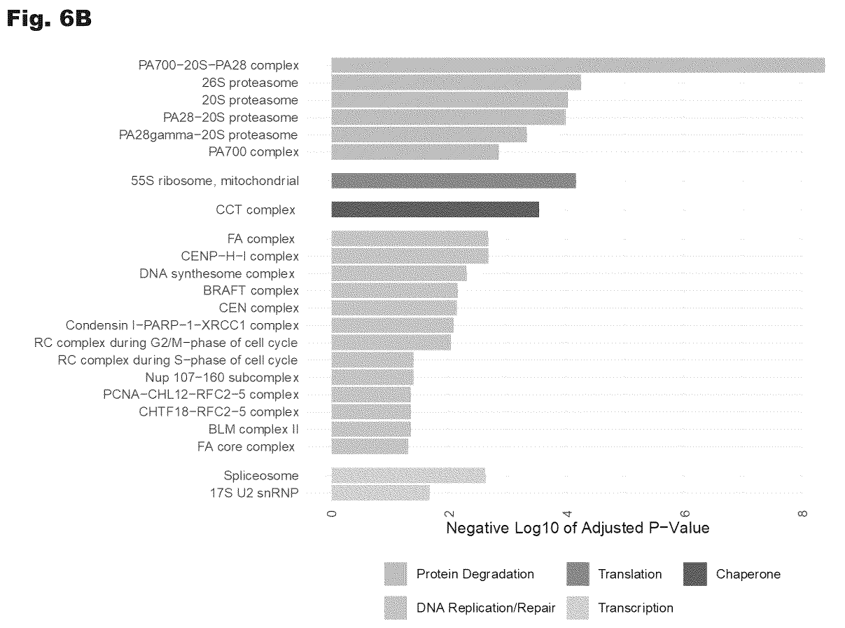

Using this approach, the GLMM was applied to all tumors in TCGA and identified 5,330 genes that are significantly up-regulated in response to mutational load (β₁>0, FDR<0.05). Next, these genes were linked to cellular function by performing gene set enrichment to known protein complexes (CORUM database) and pathways (KEGG database) using gprofiler2 (FIG. 5). As expected for tumors with many mutations, pathways and protein complexes related to cell cycle, DNA replication and DNA repair were enriched in tumors with a high mutational load. However, some of the most significant enrichment terms were for protein complexes and pathways that regulate translation (mitochondrial ribosomes), protein degradation (proteasome complex), and protein folding (CCT complex/HSP60), consistent with the hypothesis that high mutational load tumors experience protein misfolding stress. Surprisingly, it was also found that the spliceosome, a large protein complex that regulates alternative splicing in cells, is up-regulated in response to mutational load. This suggests that transcription itself could also be regulated in response to protein misfolding stress. In addition, it was confirmed that the same proteostasis complexes are identified when performing gene set enrichment analysis only genes with the largest effect sizes from the transcriptional screen (in the upper quartile of β₁ regression coefficients), which contain half the number of significant genes as identified previously (N=2,152 vs 5,330; FIGS. 6A and 6B).

### Gene Silencing Through Alternative Splicing in High Mutational Load Tumors.

It was next investigated in detail how these protein complexes could mitigate the damaging effects of protein misfolding in high mutational load tumors by examining the role of the spliceosome in gene silencing. It was hypothesized that the up-regulation of the spliceosome in high mutational load tumors prevents further protein misfolding by regulating pre-mRNA transcripts to be degraded rather than translated. The down-regulation of gene expression via alternative splicing events, such as intron retention, is one mechanism to silence genes by funneling transcripts to mRNA decay pathways.

To test whether gene expression is down-regulated in high mutational load tumors through intron retention, previously called alternative splicing events in TCGA were utilized. Alternative splicing events within this dataset were quantified through a metric called percent spliced in or PSI. PSI is calculated as the number of reads that overlap the alternative splicing event (e.g. for intron retention, either at intronic regions or those at the boundary of exon to intron junctions) divided by the total number of reads that support and don't support the alternative splicing event. Thus, PSI estimates the probability of alternative splicing events only at specific exonic boundaries in the entire transcript population without requiring information on the complete underlying composition of each full length-transcript.

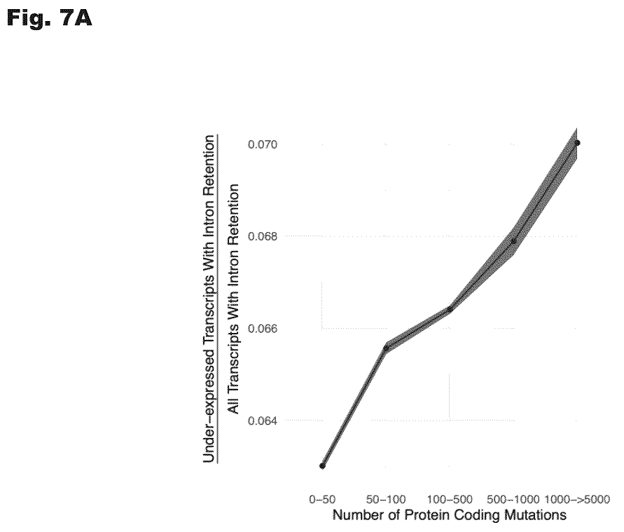

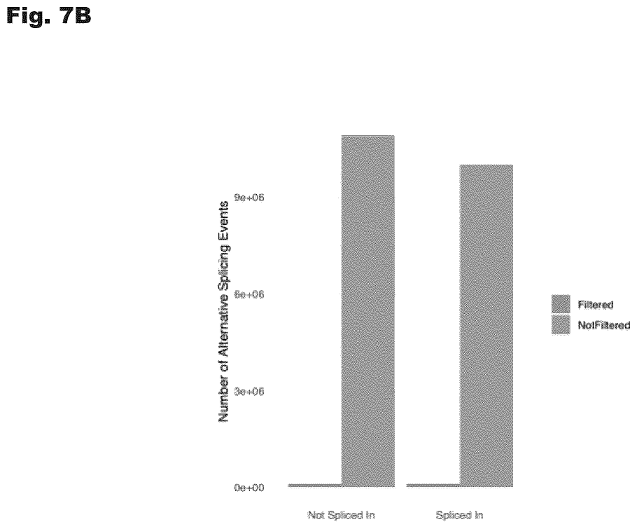

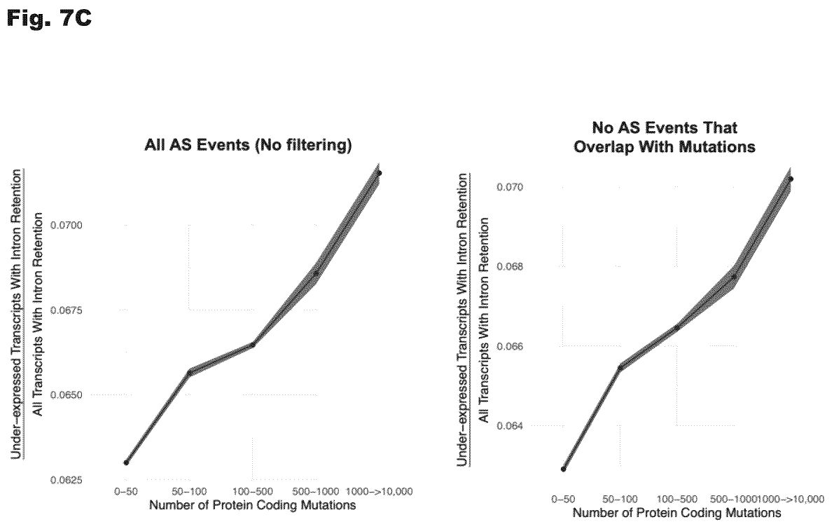

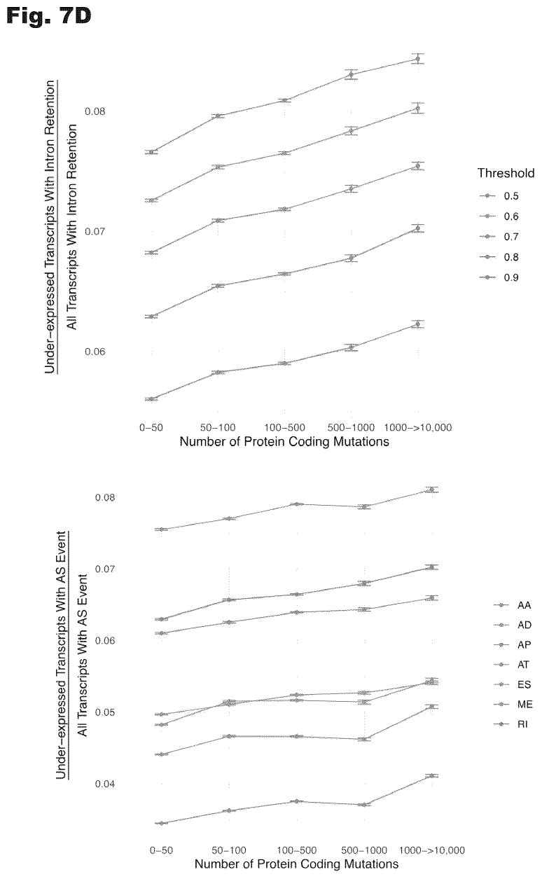

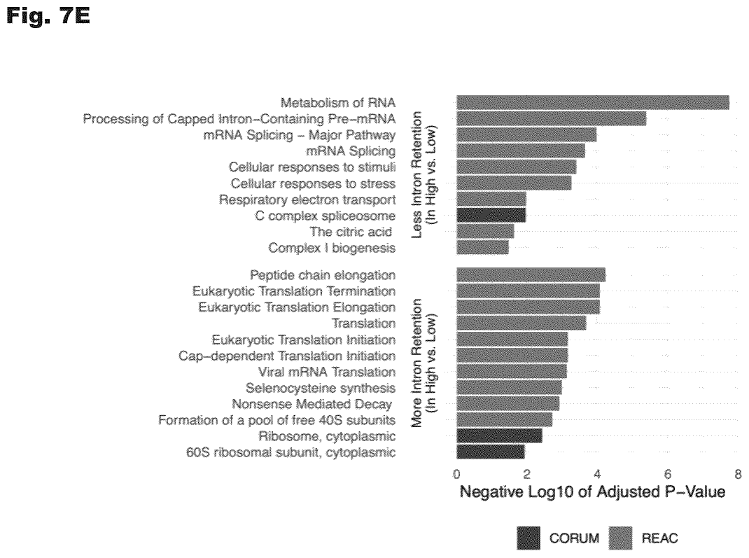

Using these alternative splicing calls, it was reasoned that if a transcript contains an intron retention event and is downregulated in expression, the transcript is more likely to have been degraded by mRNA decay pathways. For all genes, it was first quantified whether intron retention events were present based on a threshold value >80% PSI. For each gene with an intron retention event, it was quantified whether the expression of the same gene was under-expressed. Each gene was counted as under-expressed if it was one standard deviation below the mean expression within the same cancer type. To control for mutations that might affect patterns of expression, (i.e., expression quantitative trait loci or eQTL effects), alternative splicing events that contained a point mutation within the same gene were removed from the analysis (which only represent ˜1% of intron retention events across all tumors; for more details, see Methods section below). It was found that relative to all transcripts with intron retention events, the number of transcripts that are under-expressed increases with tumor mutational load (FIG. 7A), suggesting that the degree of intron-retention driven mRNA decay is elevated in high mutational load tumors. This trend is robust to other PSI value thresholds (>50-90% PSI), even for other alternative splicing events (e.g., exon skipping, mutually exclusive exons, etc.) and when not filtering for potential eQTL effects (Supplemental FIGS. 7B, 7C and 7D).

It was next investigated which genes are more likely to be silenced through mRNA decay between low and high mutational load tumors. For each intron retention event, it was calculated whether PSI values were significantly different in low mutational load tumors (<10 total protein-coding mutations) compared to high mutational load tumors (>1000 total protein-coding mutations) using a t-test. This approach identified 606 and 201 genes that have more and less intron retention events in high mutational load tumors, respectively. Using gene set enrichment analysis, it was found that cytoplasmic ribosomes contain more intron retention events in high mutational load tumors, potentially leading to their down-regulation through mRNA decay to prevent further protein mis-folding (FIG. 7E). Genes that contain fewer intron retention events in high mutational load tumors, which are less likely to undergo mRNA decay, are primarily related to mRNA splicing.

### Regulation of Translation, Protein Folding and Protein Degradation in High Mutational Load Tumors.

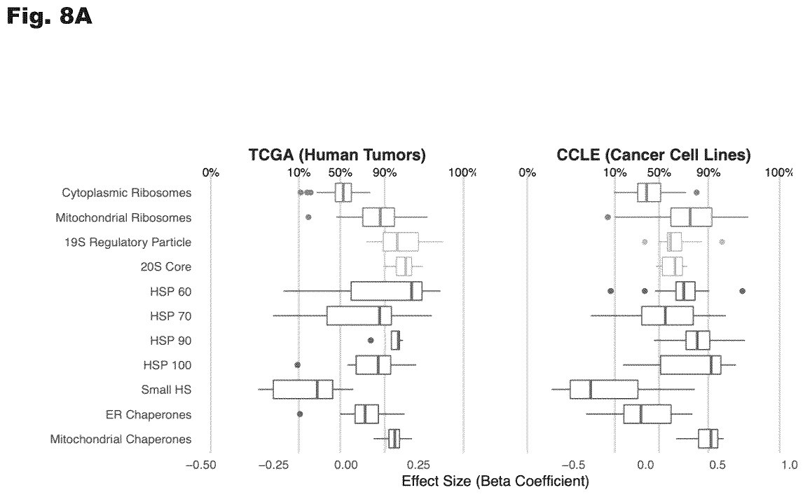

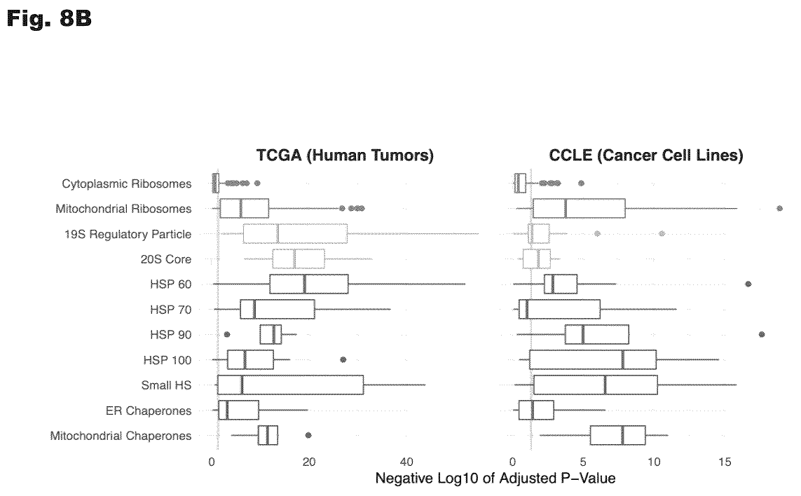

It was next investigated in detail how the remaining proteostasis complexes that were significant in the genome-wide screen, which regulate protein synthesis, degradation and folding, could mitigate protein misfolding in high mutational load tumors. To do so, we expanded our gene sets to include other chaperone families, all ribosomal complexes and proteasomal subunits (FIGS. 8A and 8B). Using the GLMM framework detailed above, it was found that the expression of nearly all individual genes in chaperone families that participate in protein folding (HSP60, HSP70 and HSP90), protein disaggregation (HSP100), and have organelle-specific roles (ER and mitochondrial) are significantly up-regulated in response to mutational load. Interestingly, however, small heat shock proteins, which don't participate in protein folding or disaggregation, are significantly down-regulated in response to increased protein coding mutations. The role of small heat shock proteins is primarily to hold unfolded proteins in a reversible state for re-folding or degradation by other chaperones and thus, could possibly be down-regulated due to their inefficiency in mitigating protein misfolding.

Differences in expression of different structural components of the proteasome were further examined, which is a large protein complex responsible for degradation of intracellular proteins. Consistent with the over-expression of chaperone families that mitigate protein mis-folding, both the 19s regulatory particle (which recognizes and imports proteins for degradation) and the 20s core (which cleaves peptides) of the proteasome are up-regulated in response to mutational load in TCGA (FIGS. 8A and 8B). In addition, it was found that specifically mitochondrial—but not cytoplasmic—ribosome complexes are up-regulated in high mutational load tumors. Mitochondrial ribosome biogenesis has been shown to occur under conditions of chronic protein misfolding as a mechanism of compartmentalization and degradation of proteins. In contrast, translation of proteins through cytosolic ribosome biogenesis has been previously characterized to be attenuated and slowed to prevent further protein mis-folding. This decrease in expression of cytoplasmic ribosomes is also consistent with observed patterns of alternative splicing coupled to mRNA decay pathways in high mutational load tumors (FIG. 7E).

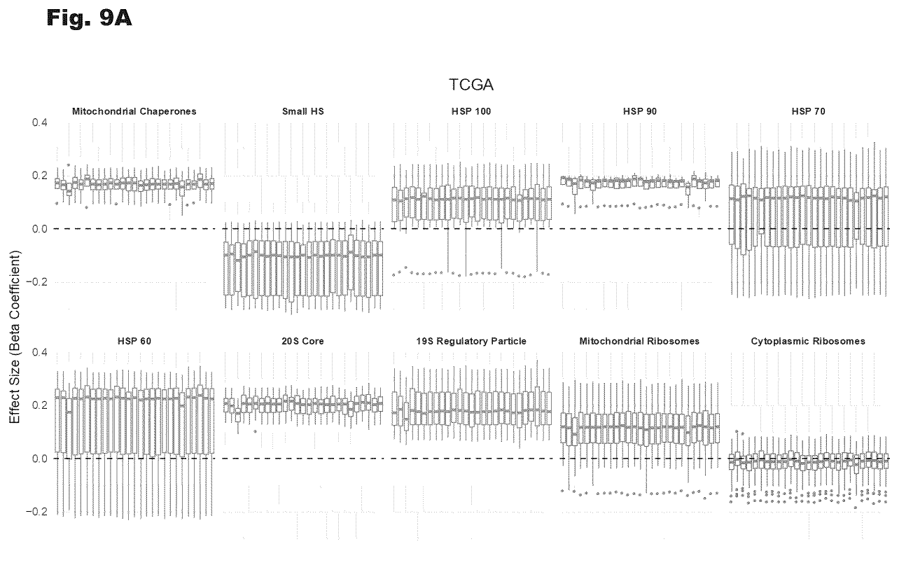

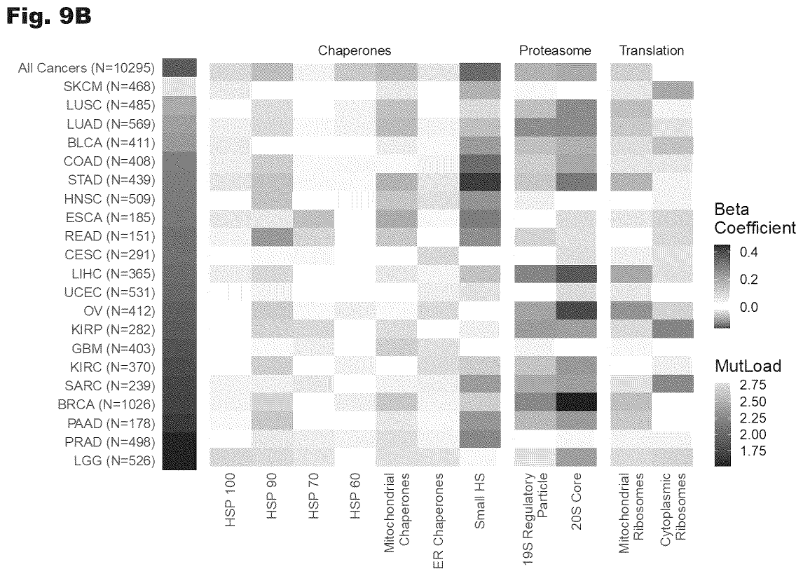

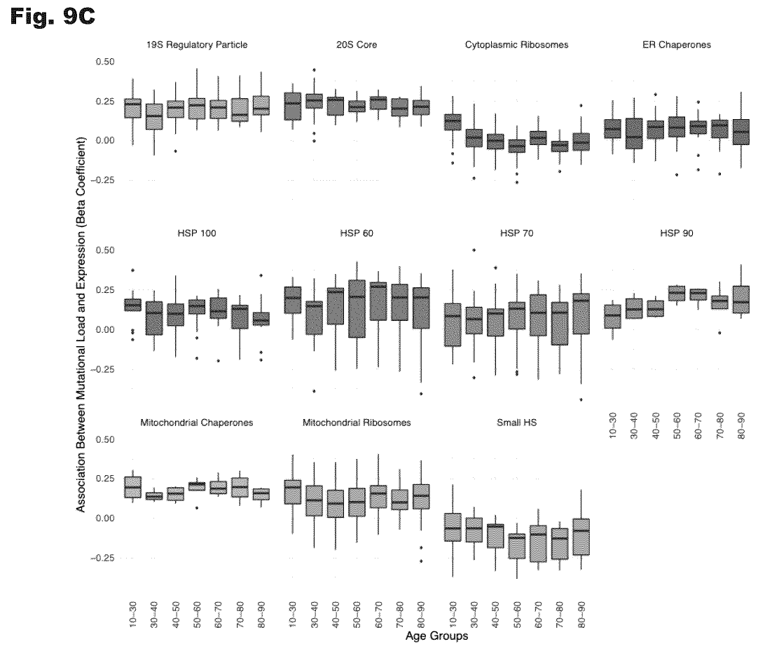

Finally, a jackknife re-sampling procedure was performed to confirm that specific cancer types were not driving patterns of association within the GLMM. This was achieved by removing each cancer type from the regression model one at a time, and re-calculating regression coefficients on the remaining set of samples. Overall, regression coefficients were stable across cancer types and trends were unchanged (FIG. 9A). In addition, linear regression was also performed within cancer types and similar expression responses to mutational load across proteostasis complexes were found FIG. 9B). Finally, it was also confirmed that patient age was not driving patterns of association of mutational load and gene expression within the GLMM (FIG. 9C). Taken together, this suggests that protein re-folding, protein disaggregation, protein degradation, and down-regulation of cytoplasmic translation are potential mechanisms to mitigate and prevent protein misfolding in high mutational load tumors.

### Validating Proteostasis Expression Responses in Cancer Cell Lines and Establishing a Causal Connection Through Perturbation Experiments.

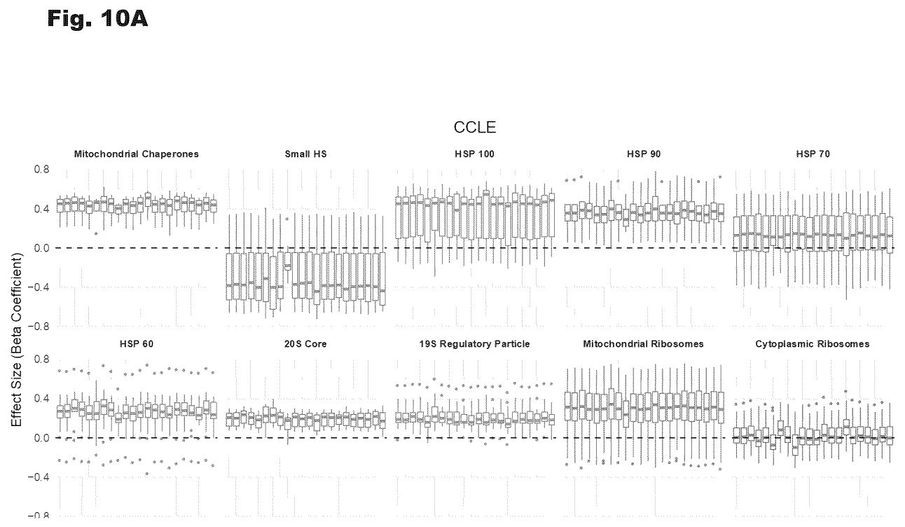

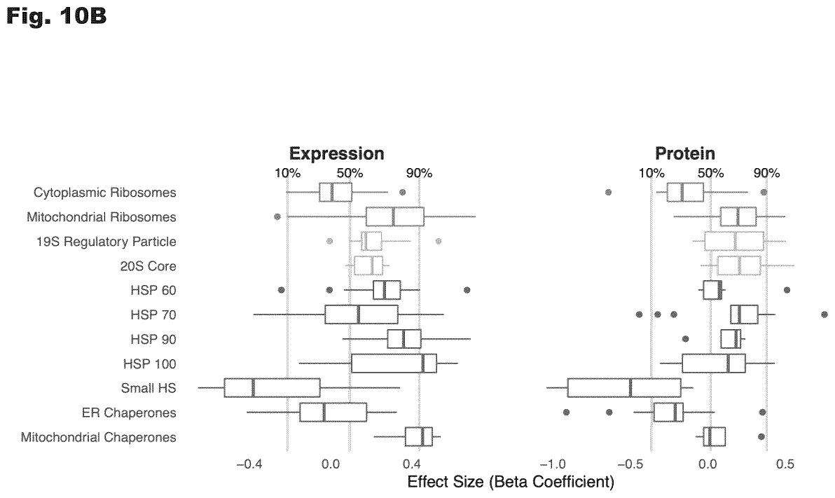

It was next sought to validate these results by first examining whether the expression patterns observed in human tumors replicate within cancer cell lines from the Cancer Cell Line Encyclopedia (CCLE). Unlike TCGA, samples within each cancer type in CCLE can be small and are unbalanced (i.e., some cancer types have <10 samples and others have >100 samples). Since GLMMs may not be able to estimate among-population variance accurately in these cases, a simple generalized linear model (GLM) was utilized instead to measure the effect of mutational load on patterns of expression without over-constraining the model. Indeed, it was found that expression patterns seen in human tumors broadly replicate in cancer cell lines (FIG. 10A). Similar to the expression analysis in TCGA, a jackknife re-sampling procedure was utilized to confirm that specific cancer types aren't driving patterns of association within the GLM (FIGS. 10A & 10B). Finally, these trends were further validated by incorporating protein abundance estimates in CCLE, which contains the largest dataset available of RNA (n=1377) and protein (n=373) abundances that are harmonized across samples. similar patterns of expression and protein abundances in response to mutational load in CCLE within proteostasis complexes were found (FIG. 10B).

Overall, this indicated that the expression patterns observed are cell autonomous (i.e., independent of organismal effects such as the immune system, age or microenvironment) and consistent across high mutational load cancer cells. Importantly, it also demonstrates that cancer cell lines are a reasonable model to causally interrogate these effects further through functional and pharmacological perturbation experiments.

To establish a causal relationship between the over-expression of proteostasis machinery and maintenance of cell viability under high mutational load, expression knock-down (shRNA) estimates from project Achilles were utilized for the same cancer cell lines as in CCLE. It was sought to measure how mutational load impacts cell viability when protein complexes and gene families undergo a loss of function through expression knock-down. Since the shRNA screen was performed on an individual gene basis, a GLM framework was utilized that aggregates expression knock-down estimates of all genes within a given proteostasis gene family to jointly measure how mutational load impacts cell viability after loss of function. Specifically, an additional categorical variable of the gene name was included within each gene family to allow for a change in the intercept within each gene in the GLM when measuring the association of mutational load and cell viability after expression knock-down. In addition, it was similarly evaluated whether specific cancer types were driving patterns of association within the GLM through jackknife re-sampling by cancer type (FIG. 11A).

Overall, it was found that elevated mutational load is associated with decreased cell viability when the function of most chaperone gene families are disrupted through expression knock-down (FIG. 11A). However, only chaperones within the HSP100 family, which have the unique ability to rescue and reactivate existing protein aggregates in cooperation with other chaperone families, show a significant negative relationship between mutational load and cell viability across almost all cancer types. Similarly, the results indicate specificity in the vulnerability that mutational load generates when the function of the proteasome and different ribosomal complexes are disrupted (FIG. 11A). Mutational load significantly decreases cell viability only when expression knock-down of the 19s regulatory particle of the proteasome is disrupted, suggesting that targeting the protein import machinery of the proteasome is more effective than targeting the protein cleaving machinery in the 20s core. Finally, mutational load significantly increases cell viability when cytoplasmic ribosomes—which are already down-regulated in response to mutational load—undergo a loss of function through expression knock-down. Conversely, expression knock-down of mitochondrial ribosomes significantly decreases viability with increased mutational load in cell lines, which is also consistent with the patterns of expression observed.

Since functional redundancy in the human genome can make expression knock-down estimates within individual genes noisy, it was also examined how drugs targeting the function of whole complexes impacts viability with mutational load across all cancer types and when removing individual cancer types through jackknife re-sampling. To do so, drug sensitivity screening data from project PRISM within CCLE was utilized and a simple GLM was used to measure the association of mutational load and cell viability after drug inhibition. It was found that treatment with the majority of proteasome inhibitors (6/8) and ubiquitin-specific proteasome inhibitors (2/3), which target protein degradation complexes, are significantly associated with a decrease in cell viability in high mutational load cell lines (FIG. 11B). Similarly, most HSP90 inhibitors decrease cell viability with mutational load (8/10), although only a few drugs show a significant relationship (FIG. 11B). This variability in the efficacy of drugs with similar mechanisms of action likely reflects that the efficacy to disrupt the function of proteostasis machinery is dependent on the specific molecular affinity of a compound to its target and downstream effectors. While these are the only relevant proteostasis drugs in the PRISM dataset that are currently available, other drugs targeting other chaperone machinery or splicing complexes would also target other vulnerabilities in high mutational load cancers. Collectively, these results indicate that elevated expression of protein degradation and folding machinery is causally related to the maintenance of viability in high mutational load cell lines, and likely in high mutational load tumors by extension.

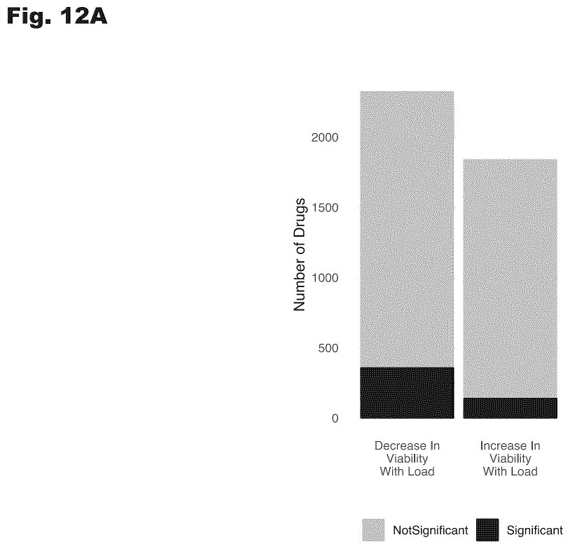

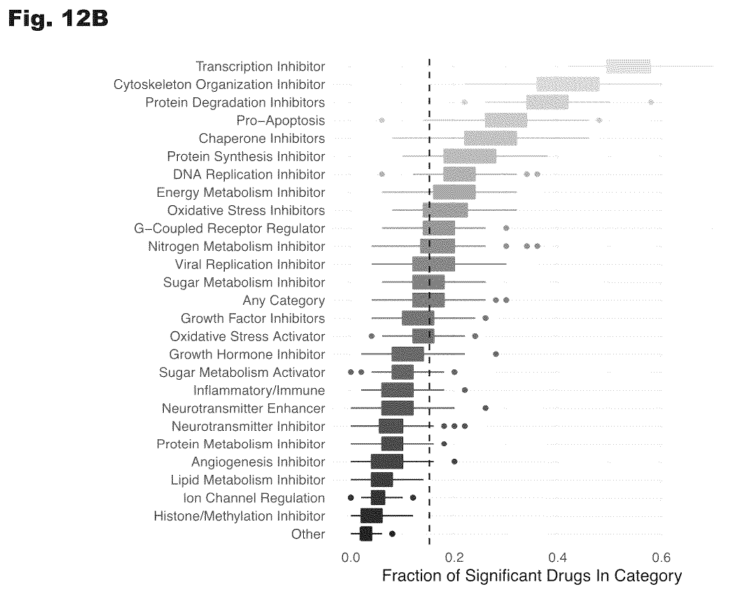

Lastly, it was found that most drugs in the PRISM database do not significantly decrease cell viability with mutational load (FIG. 12A), suggesting that high mutational load cancer cells are not generically vulnerable to all classes of drugs. Specifically, it was found that drugs which inhibit transcription, cytoskeleton organization, protein degradation, chaperones, protein synthesis and promote apoptosis are most effective at targeting high mutational load cancer cells—delineating additional potential therapeutic vulnerabilities in high mutational burden tumors (FIG. 12B).

### Methods

Data availability and resources. Whole-exome, somatic mutation calls of 10,486 cancer patients across 33 cancer types in The Cancer Genome Atlas (TCGA) were downloaded from the Multi-Center Mutation Calling in Multiple Cancers (MC3) project (Ellrott, K. et al. Cell Syst. 6, 271-281.e7 (2018), the disclosure of which is incorporated herein by reference; gdc.cancer.gov/about-data/publications/mc3-2017). For the same patients in TCGA, RNA-seq data of log₂ transformed RSEM normalized counts were downloaded from the UCSC Xena Browser (Goldman, M. J. et al. Nature Biotechnology (2020), the disclosure of which is incorporated herein by reference; xenabrowser.net/datapages/) and copy number alterations (CNAs), including amplifications and deletions, called via ABSOLUTE were downloaded from COSMIC (v91) (Bamford, S. et al. Br. J. Cancer (2004), the disclosure of which is incorporated herein by reference; cancer.sanger.ac.uk/cosmic/download). Tumor purity estimates for TCGA were downloaded from the Genomic Database Commons (GDC) (Grossman, R. L. et al. N. Engl. J. Med. 375, 1109-1112 (2016), the disclosure of which is incorporated herein by reference; gdc.cancergov/about-data/publications/pancanatlas). Data for all cancer cell lines in the Cancer Cell Line Encyclopedia (CCLE) were downloaded from DepMap (Barretina, J. et al. Nature (2012), the disclosure of which is incorporated herein by reference; depmap.org/portal/download/all/). Specifically, mutation calls (Version 21Q3) from whole-exome sequencing data, copy number alternations quantified by ABSOLUTE (Version CCLE 2019), log₂ transformed TPM normalized counts (Version 21Q3) from RNA-seq data, proteomics data quantified by mass spectrometry, shRNA data from project Achilles (Tsherniak, A. et al. Cell (2017), the disclosure of which is incorporated herein by reference) normalized using DEMETER (DEMETER2 Data v6), and primary drug sensitivity screens of replicate collapsed log fold changes relative to DMSO from project PRISM (Version 19Q4; Corsello, S. M. et al. Discovering the anticancer potential of non-oncology drugs by systematic viability profiling. Nat. Cancer (2020), the disclosure of which is incorporated herein by reference) were used.

Statistical analysis. The ImerTest and Imer package in R was used to apply a separate generalized linear mixed model (GLMM) for each gene in the genome to identify groups of genes whose expression is up-regulated in response to mutational load in TCGA. For each gene, expression values across all patients were z-score normalized in all analyses to ensure fair comparisons across genes. Known co-variates of tumor purity and cancer type were included in the GLMM. Tumor purity and mutational load were modeled as fixed effects, whereas cancer type was modeled as a random effect (i.e. random intercept) since it varies across groups of patients and can be interpreted as repeated measurements across groups. For all analyses, mutational load was defined as login of the number of synonymous, nonsynonymous and nonsense mutations per tumor. For each gene, the parameters used in the GLMM were as follows,

Y˜β₀+β₁X₁+β₂X₂+v+e

where Y is a vector of expression values of each tumor, β₀ is the fixed intercept, β₁ is the fixed slope for the predictor variable X₁ which is a vector of mutational load values for each tumor, β₂ is the fixed slope for the predictor variable X₂ which is a vector of the purity of each tumor, v is the random intercept for each cancer type, and e is a Gaussian error term. To examine expression responses to mutational load within a given protein complex and cancer type, the same normalization procedures were applied as above within cancer types and a separate GLM for each cancer type was ran as follows,

Y˜β₀+β₁X₁+β₂X₂+β₃X₃+e

where Y is a vector of expression values of each tumor in a given cancer type, β₀ is the fixed intercept, β₁ is the fixed slope for the predictor variable X₁ which is a vector of mutational load values for each tumor, β₂ is the fixed slope for the predictor variable X₂ which is a vector of the purity of each tumor, β₃ is a change in the intercept for X₃ which is a categorical variable of individual genes within each proteostasis complex and e is a Gaussian error term.

Unlike TCGA, samples within each cancer type in CCLE can be small and are unbalanced (i.e. some cancer types have <10 samples and others have >100 samples). In these cases, mixed effects models may not be able to estimate among-population variance accurately. Thus, for all regression-based analyses in CCLE, a simple generalized linear model (GLM) was used instead. Cell viability values across all cell lines were z-score normalized by gene in all analyses to ensure fair comparisons across genes. To assess whether the same sets of genes are up-regulated in response to mutational load in CCLE using the GLM, a similar procedure to the GLMM was performed. A separate GLM was applied for each gene with the following parameters

Y˜β₀+β₁X₁+e

where Y is a vector normalized expression values of each cell line, β₀ is the fixed intercept, β₁ is the fixed slope for the predictor variable X₁ which is a vector of mutational load values for each tumor, and e is a Gaussian error term. To assess whether protein abundances are similarly up-regulated in response to mutational load in CCLE in proteostasis complexes, a separate GLM was applied to each gene with the following parameters,

Y˜β₀+β₁X₁+β₂X₂+e

where Y is a vector of normalized cell viability estimates after expression knock-down of an individual gene across all cell lines, β₀ is the fixed reference intercept, β₁ is the fixed slope for the predictor variable X₁ which is a vector of mutational load values for each cell line, β₂ is a change in the intercept for X₂ which is a categorical variable of individual genes within each proteostasis complex, and e is a Gaussian error term. To estimate the association of mutational load and cell viability after pharmacologic inhibition of proteostasis machinery, the following GLM was applied to each relevant drug in PRISM:

Y˜β₀+β₁X₁+e

where Y is a vector normalized cell viability estimates after drug inhibition across all cell lines, β₀ is the fixed intercept, β₁ is the fixed slope for the predictor variable X₁ which is a vector of mutational load values for each tumor, and e is a Gaussian error term.

Model validation. For both the GLM and GLMM, model assumptions of homogeneity of variance were verified by plotting residuals versus fitted values in the model and residuals versus each covariate in the model. Multi-collinearity with other mutational classes (e.g. such as copy number alterations, CNAs) were considered but not found to correlate with point mutations (FIG. 3). A jackknife re-sampling procedure was used for outlier analysis and to determine whether specific cancer types are driving patterns of association within the GLM and GLMM. Briefly, each cancer type was removed from the regression model one at a time, and regression coefficients were re-estimated. Overall, regression coefficients were fairly stable across cancer types and trends remained the same (FIGS. 9A, 10A and 10B).

Proteostasis gene sets. Genes for chaperone complexes were identified from 76 and genes that are co-chaperones were not considered. Proteasome and ribosomal complexes were identified from CORUM (Giurgiu, M. et al. Nucleic Acids Res. (2019), the disclosure of which is incorporated herein by reference).

Gene set enrichment analysis. All gene set enrichment analysis was performed using gprofiler2 with default parameters (Peterson, H., et al. F1000Research (2020), the disclosure of which is incorporated herein by reference). For all sets of genes, significance was determined after correcting for multiple hypothesis testing (FDR<0.05). For gene set enrichment analysis used to identify genes up-regulated in TCGA in response to mutational load, all terms in CORUM database were reported and enrichment terms in the KEGG database of diseases not related to cancer (e.g. ‘Influenza A’) were omitted from the main figures for clarity and space (Tanabe, M. & Kanehisa, M. Curr. Protoc. Bioinforma. (2012), the disclosure of which is incorporated herein by reference). For gene sets used to identify terms differentially splice in between high and low mutational load tumors, all terms in the CORUM and the REACTOME database were reported in the main figures.

Alternative splicing analysis. Alternative splicing events were quantified through a previously established metric called PSI. PSI is calculated as the number of reads that overlap the alternative splicing event (e.g. for intron retention, either at intronic regions or those at the boundary of exon to intron junctions) divided by the total number of reads that support and don't support the alternative splicing event. PSI summarizes alternative splicing events at specific exonic boundaries in the entire transcript population without needing to know the complete underlying composition of each full length-transcript.

Alternative splicing calls for all tumors in TCGA were downloaded from TCGA SpliceSeq (Ryan, M. et al. Nucleic Acids Res. (2016), the disclosure of which is incorporated herein by reference). Default splice event filters (percentage of samples with PSI values >75%) from the database were applied. To test whether gene expression is down-regulated in high mutational load tumors through alternative splicing, we calculated whether alternative splicing events were present based on different threshold values of percent spliced in (PSI) from 90% to 50%. (FIG. 7D). For each alternative splicing event in a gene, it was quantified whether the expression of the same gene was under-expressed. Each gene was counted as under-expressed if it was one standard deviation below the mean expression within each cancer type. Genes that contained a point mutation within the same alternative splicing event were removed to control for eQTL effects. Intron retention events removed from this analysis represent only ˜1% of intron retention events across all tumors and similar trends are found when this filtering scheme is not applied (FIGS. 7B and 7C). In addition, it was evaluated whether this trend is robust to other alternative splicing events (i.e., Alternate Donor Sites, Alternate Promoters, Alternate Terminators, Exon Skipping Events, ME=Mutually Exclusive Exon; FIG. 7D).

To investigate which genes are differentially spliced in between low and high mutational load tumors for specific alternative splicing events (i.e. intron retention), a t-test was used to calculate whether PSI values were significantly different in tumors with <10 protein-coding mutations compared to tumors with >1000 protein-coding mutations. Each alternative splicing event within a gene was required to have less than 25% of missing PSI values and a mean difference between the two groups of >0.01 to be considered. This approach identified 606 and 201 significant genes that have more and fewer intron retention events in high mutational load tumors, respectively, after correcting for multiple hypothesis testing (FDR<0.05).

Drug category annotation and enrichment analysis. A separate GLM was ran for all drugs in the PRISM database to evaluate whether they are associated with mutational load and cell viability. All drugs that were negatively associated with mutational load and viability were queried on PubMed based on their reported mechanism of action in PRISM and grouped into broad categories (Table 1). Categories of drug mechanism of action were first chosen based on their role in metabolism and known hallmarks of cancer. Additional categories not directly related to known cancer associated functional groups were made for drugs that could not otherwise be grouped (i.e. ‘Ion Channel Regulation’, Viral Replication Inhibitor’, etc.). Drugs with ambiguous mechanism of action (e.g. ‘cosmetic’, ‘coloring agent’) were grouped into ‘Other’. The abstracts of up to 10 associated papers were used to examine for evidence connecting drug mechanisms of action to 33 broad categories. In total, 700 drug mechanism of action were grouped and annotated into 33 broad categories. These broad categories were used to assess whether high mutational load cancer cell lines are generically vulnerable to drugs or whether certain categories are more likely to contain drugs effective against high mutational load cell lines. To control for differences in the number of drugs within each category, 50 drugs were randomly sampled, and the fraction of drugs significantly associated with mutational load in each category was calculated 100 times to generate confidence intervals.

What is claimed is:

1. A method of determining a treatment regimen for a neoplasm or cancer, comprising:
assessing genetic material of a neoplasm or cancer of an individual to determine a mutational burden;
based on an amount of mutational burden, determine a treatment regimen.

2. The method of claim 1, wherein assessing genetic material comprises quantifying the amount of somatic mutations within the genetic material.

3. The method of claim 2, wherein the somatic mutations comprise single nucleotide variations (SNVs), copy number variations (CNVs), insertions, and deletions.

4. The method of claim 2 further comprising:
performing a high-throughput sequencing reaction on the genetic material of the neoplasm or the cancer to yield a sequencing result;
aligning the sequencing result of the neoplasm or the cancer against a reference genome to identify genetic variations within the genetic material of the neoplasm or the cancer, wherein the genetic variations within the genetic material of the neoplasm or the cancer comprises somatic mutations.

5. The method of claim 4 further comprising:
performing a high-throughput sequencing reaction on genetic material of a control sample to yield a sequencing result;
aligning the sequencing result of the control sample against a reference genome; and
aligning the sequencing result of the neoplasm or the cancer with the sequencing result of the control sample to identify somatic variations within the neoplasm or the cancer.

6. The method of claim 4, wherein the high-throughput sequencing is whole genome sequencing, whole exome sequencing, or targeted sequencing.

7. The method of claim 1 further comprising:
obtaining a biopsy of the individual, wherein the biopsy is a tumor excision, a liquid biopsy, or a biological waste biopsy; and
extracting the genetic material of the neoplasm or the cancer from the biopsy.

8. The method of claim 1 further comprising:
when it is determined that the amount of mutational burden is greater than a threshold, administering to the individual the treatment regimen.

9. The method of claim 2, wherein the threshold is a mutational burden in the top 25% of a particular cancer type or is a mutational burden in the top 25% of all cancer types.

10. The method of claim 2, wherein the threshold is a mutational burden in the top 10% of a particular cancer type or is a mutational burden in the top 10% of all cancer types.

11. The method of claim 2, wherein the threshold is a mutational burden in the top 5% of a particular cancer type or is a mutational burden in the top 5% of all cancer types.

12. The method of claim 8, wherein the treatment regimen comprises administration of a HSP90 inhibitor, wherein the HSP90 inhibitor is alvespimycin, BIIB021, CCT018159, ganetespib, gedunin, NVP-AUY922, PU-H71, or VER-49009.

13. The method of claim 8, wherein the treatment regimen comprises administration of a proteasome inhibitor, wherein the proteasome inhibitor is bortezomib, carfilzomib, delanzomib, ixazomib, ixazomib-citrate, MG-132, ONX-0914, or oprozomib.

14. The method of claim 8, wherein the treatment regimen comprises administration of a ubiquitin-specific proteasome inhibitor, wherein the ubiquitin-specific proteasome inhibitor is NSC-632839, P22077, or P5091.

15. The method of claim 1, wherein the neoplasm or the cancer is bile duct cancer, bladder cancer, bone cancer, brain cancer, breast cancer, colon/colorectal cancer, endometrial/uterine cancer, esophageal cancer, gall bladder cancer, gastric cancer, head and neck cancer, kidney cancer, liver cancer, lung cancer, neuroblastoma, ovarian cancer, pancreatic cancer, prostate cancer, rhabdoid tumor, sarcoma, skin cancer, or thyroid cancer.

16. A method of assessing cytotoxicity of a compound on a neoplastic cell, a cancer cell, or a tumor cell with high mutation burden, comprising:
performing a high-throughput sequencing reaction on genetic material of a specimen to yield a sequencing result, wherein the specimen is a growth of neoplastic cells, a growth of cancer cells, or a tumor;
quantifying the amount of somatic mutations within the genetic material;
determining that the specimen has a mutational burden that is greater than a threshold;
contacting a neoplastic cell of the growth of neoplastic cells, a cancer cell of the growth of neoplastic cells, or a tumor cell of the tumor with a compound to assess the cytotoxicity of the compound on the neoplastic cell, the cancer cell, or the tumor cell.

17. The method of claim 16, wherein the neoplastic cell, the cancer cell, or the tumor cell is in vitro.

18. The method of claim 16, wherein the neoplastic cell, the cancer cell, or the tumor cell is in vivo.

19. The method of claim 15, wherein the compound is classified as: a transcription inhibitor, a cytoskeleton organization inhibitor, a protein degradation inhibitor, an agonist of apoptosis, a chaperone inhibitor, a protein inhibitor, a DNA replication inhibitor, an energy metabolism inhibitor, an oxidative stress inhibitor, a G-coupled receptor modulator, a HSP90 inhibitor, a proteasome inhibitor, or a ubiquitin-specific proteasome inhibitor.

20. The method of claim 15, wherein the somatic mutations comprise at least one of: single nucleotide variations (SNVs), copy number variations (CNVs), insertions, or deletions.
